(19) United States
(12) Patent Application Publication
(10) Pub. No.: US 2018/0362580 A1
(43) Pub. Date: Dec. 20, 2018

(54) CRYSTALLINE AND AMORPHOUS FORMS OF CARFILZOMIB

(71) Applicant: Mylan Laboratories Limited, Hyderabad (IN)

(72) Inventors: Vinayak Gore, Hyderabad (IN); Vinay Kumar Shukla, Hyderabad (IN); Yogesh Sangvikar, Hyderabad (IN); Dattatrey Kokane, Hyderabad (IN); Sushant Gharat, Hyderabad (IN)

(73) Assignee: Mylan Laboratories Limited, Hyderabad (IN)

(21) Appl. No.: 16/060,774

(22) PCT Filed: Dec. 10, 2016
(86) PCT No.: PCT/IN2016/050439

§ 371 Date: Jun. 8, 2018

(30) Foreign Application Priority Data
Dec. 11, 2015 (IN) 6647/CHE/2015

(51) Int. Cl. C07K 5/103; A61K 47/40

(52) U.S. Cl. CPC C07K 5/1008; A61K 38/00; C07B 2200/13; A61K 47/40

(57) ABSTRACT

The present disclosure provides crystalline carfilzomib form M1 and a process for the preparation thereof. Further disclosed are processes for the preparation of amorphous carfilzomib using crystalline form M1 as a starting material. The present disclosure also relates to an improved process for the preparation of carfilzomib.

### CROSS-REFERENCE TO RELATED APPLICATIONS

This application claims the benefit of earlier Indian provisional patent application no. 6647/CHE/2015, filed on Dec. 11, 2015, which is incorporated by reference herein in its entirety.

### FIELD OF THE INVENTION

The present disclosure relates to crystalline carfilzomib form M1 and preparation of amorphous carfilzomib using crystalline carfilzomib form M1.

### BACKGROUND OF THE INVENTION

Carfilzomib is a proteasome inhibitor, marketed as KYPROLIS® by Amgen. KYPROLIS® is indicated for treatment of multiple myeloma. Carfilzomib is chemically known as (2S)-N-((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide and structurally represented as Formula I below.

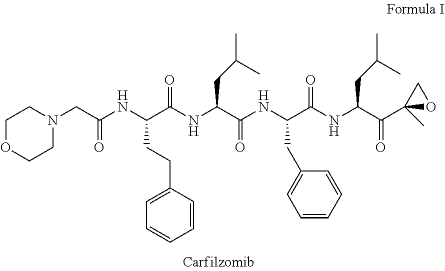 [SMILES: CC(C)C[C@H](NC(=O)[C@H](CCc1ccccc1)NC(=O)CN1CCOCC1)C(=O)N[C@@H](Cc1ccccc1)C(=O)N[C@@H](CC(C)C)C(=O)[C@@]1(C)CO1]

U.S. Pat. No. 7,417,042, discloses a process for the preparation of carfilzomib. U.S. Pat. No. 8,367,617, discloses crystalline carfilzomib, a citrate salt of carfilzomib, and amorphous carfilzomib.

### SUMMARY OF THE INVENTION

One aspect of the present disclosure provides crystalline carfilzomib form Ml. The crystalline carfilzomib form M1, prepared by methods disclosed herein may be characterized by a powder X-ray diffraction pattern with significant peaks at 6.33, 12.76, and 19.85±0.2° 2θ.

Within the context of the disclosure, the crystalline carfilzomib form M1 may be further characterized by a powder X-ray diffraction pattern with significant peaks at 6.33, 9.28, 12.56, 12.76, 18.67, 19.04, 19.85, and22.78±0.2° 2θ.

In another aspect, the present disclosure provides a process for preparation of crystalline carfilzomib form M1.

In one embodiment, crystalline carfilzomib form M1 may be prepared by a process that includes the following steps:
a) dissolving carfilzomib in an organic solvent at an elevated temperature to form a solution;
b) adding water to the solution;
c) cooling the solution; and
d) isolating crystalline carfilzomib form M1.

Within the context of this embodiment, examples of suitable solvents include acetonitrile, toluene, and mixtures thereof.

Within the context of this embodiment, the dissolving of the carfilzomib may be carried out at a temperature of about 35° C. to about 50° C.

In another aspect, the present disclosure provides a process for preparation of amorphous carfilzomib.

In one embodiment, amorphous carfilzomib may be prepared by a process that includes the following steps:
a) dissolving crystalline carfilzomib in an alcohol solvent to form a solution;
b) combining the solution with water;
c) cooling the solution; and
d) isolating amorphous carfilzomib.

Within the context of this embodiment, the alcohol solvent may be, for example, methanol, ethanol, isopropanol, butanol, or mixtures thereof.

Another aspect of the present invention provides a pharmaceutical composition containing amorphous carfilzomib. The pharmaceutical composition may, in some embodiments, contain one or more pharmaceutically acceptable excipients. Examples of suitable pharmaceutically acceptable excipients include cyclodextrins, such as sulfobutylether beta-cyclodextrin, hydroxypropyl-beta-cyclodextrin, and the like.

Another aspect of the present invention provides a pharmaceutical composition containing crystalline carfilzomib form M1. The pharmaceutical composition may, in some embodiments, contain one or more pharmaceutically acceptable excipients. Examples of suitable pharmaceutically acceptable excipients include cyclodextrins, such as sulfobutylether beta-cyclodextrin, hydroxypropyl-beta-cyclodextrin, and the like.

### BRIEF DESCRIPTION OF THE DRAWINGS

The present disclosure together with additional features contributing thereto and advantages accruing there from will be apparent from the following description of embodiments of the disclosure which are shown in the accompanying drawing figures wherein:

FIG. 1 is a powder X-ray diffraction (PXRD) pattern of crystalline carfilzomib form M1;

FIG. 2 is a differential scanning calorimetry (DSC) thermogram of crystalline carfilzomib form M1;

FIG. 3 is a thermogravimetric analysis (TGA) trace of crystalline carfilzomib form M1; and

FIG. 4 is a PXRD pattern of amorphous carfilzomib.

### DETAILED DESCRIPTION OF THE INVENTION

It is to be understood that the description of the present disclosure has been simplified to illustrate elements that are relevant for a clear understanding of the invention, while eliminating, for purposes of clarity, other elements that may be well known.

The present disclosure provides an improved process for preparation of carfilzomib. The present disclosure also provides crystalline carfilzomib form M1, a process for the preparation thereof, as well as a process for preparation of amorphous carfilzomib using crystalline carfilzomib form M1.

The polymorphs disclosed herein may be characterized by their X-ray powder diffraction (PXRD) patterns. Thus, samples of carfilzomib prepared by processes disclosed herein, including crystalline carfilzomib form M1 and amorphous carfilzomib, were analyzed by PXRD on a BRUKER D-8 DISCOVER powder diffractometer equipped with goniometer of θ/2θ configuration and LYNX EYE detector. The Cu-anode X-ray tube was operated at 40 kV and 30 mA. The experiments were conducted over the 2θ range of 2.0°-50.0°, 0.030° step size, and 0.4 seconds step time.

The polymorphs disclosed herein may also be characterized by differential scanning calorimetry (DSC). Thus, samples of carfilzomib prepared by processes disclosed herein, including crystalline carfilzomib form M1 and amorphous carfilzomib, were analyzed by DSC on TA Q1000 of TA instruments. The experiments were performed at a heating rate of 10° C./min over a temperature range of 30° C.-250° C. purging with nitrogen at a flow rate of 50mL/min. Standard aluminum pans covered by lids with three pin holes were used.

The polymorphs disclosed herein may also be characterized by thermogravimetric analysis (TGA). Thus, samples of carfilzomib prepared by processes disclosed herein, including crystalline carfilzomib form M1 and amorphous carfilzomib, were analyzed by TGA on a TA Q5000 IR of TA instruments. The experiments were performed at a heating rate of 10° C./min over a temperature range of ambient—300° C. purging with nitrogen at a flow rate of 25 mL/min.

In one aspect, the present disclosure provides crystalline carfilzomib form M1.

Within the context of this disclosure, crystalline carfilzomib form M1 may be characterized by a PXRD pattern having significant peaks at 6.33, 12.76, and 19.85±0.2° 2θ.

Crystalline carfilzomib form M1 may be further characterized by an X-ray powder diffraction pattern having significant peaks at 6.33, 9.28, 12.56, 12.76, 18.67, 19.04, 19.85, and 22.78±0.2° 2θ.

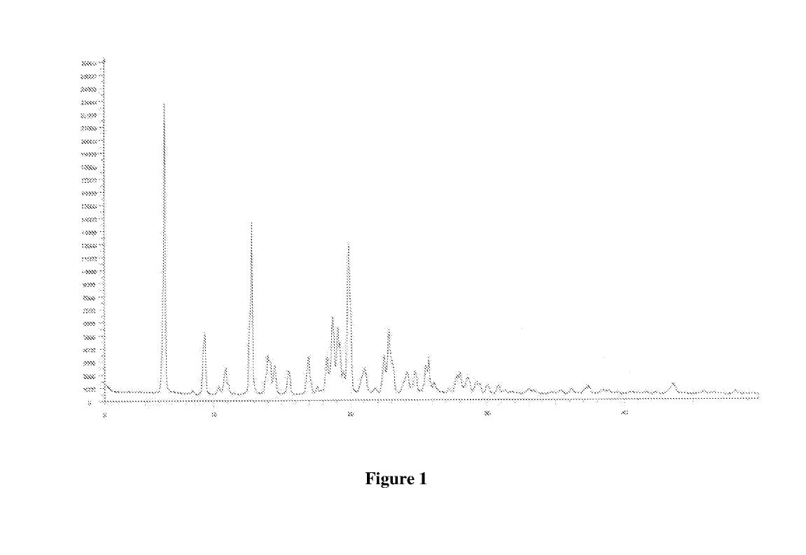

Crystalline carfilzomib form M1 may be further characterized by the PXRD pattern in FIG. 1.

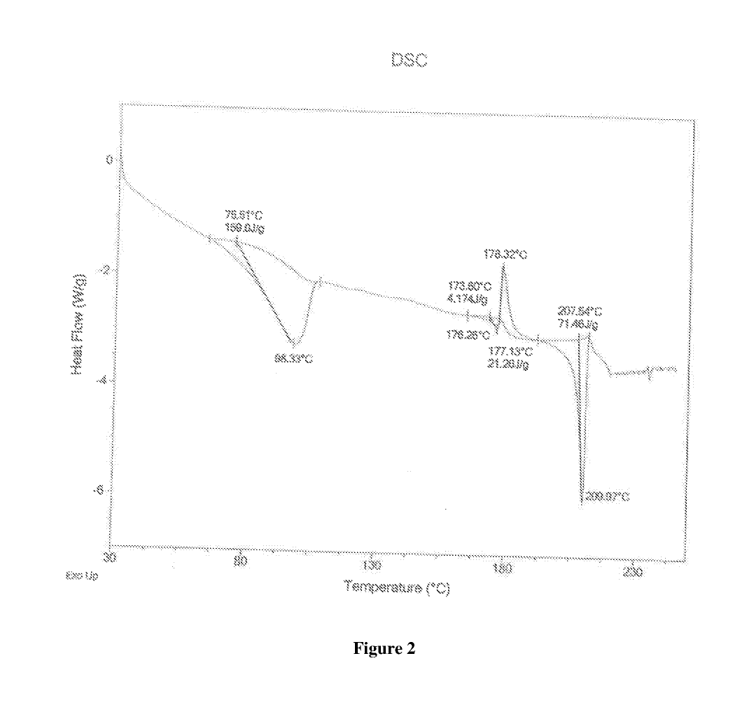

Crystalline carfilzomib form M1 may be further characterized by the DSC thermogram in FIG. 2. Without being limited by one particular theory, it is believed that the peak at 98.33° C. corresponds to evaporation of water and acetonitrile.

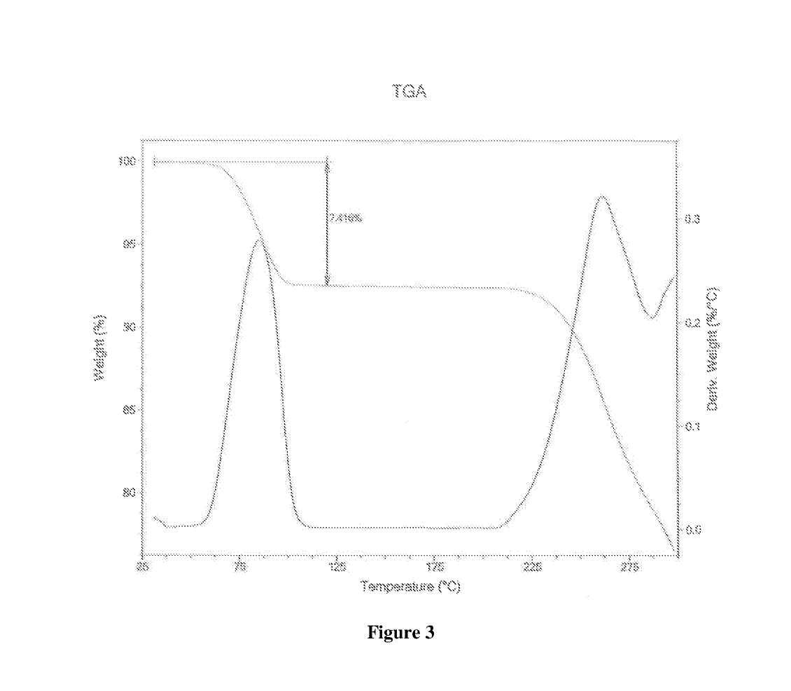

Crystalline carfilzomib form M1 may be further characterized by the TGA trace in FIG. 3. It is believed that loss of 7.416% corresponds to loss of water and acetonitrile.

In another aspect, the present disclosure provides a process for the preparation of crystalline carfilzomib form M1.

In one embodiment, crystalline carfilzomib form M1 may be prepared by a process that includes the following steps:
a) dissolving carfilzomib in an organic solvent at an elevated temperature to form a solution;
b) adding water to the solution;
c) cooling the solution; and
d) isolating crystalline carfilzomib form M1.

According to this embodiment, carfilzomib may be dissolved in a solvent an elevated temperature. For example, the temperature may be 35° C. to 50° C. In some embodiments, the temperature is about 40° C. to about 45° C.

Within the context of the invention, the term “about” when modifying a temperature measurement is meant to mean the recited temperature plus or minus five degrees. Within the context of the invention, the term “about” when modifying an absolute measurement, such as time, mass, or volume, is meant to mean the recited value plus or minus 10% of that value.

Within the context of this embodiment, the solvent may be, for example, acetonitrile, toluene, or mixtures thereof. In some particularly useful embodiments, acetonitrile is used.

Within the context of this embodiment, the carfilzomib starting material may be any form of carfilzomib, for example, any crystalline form, a salt of carfilzomib, a solvate of carfilzomib, or amorphous carfilzomib.

Next, water may be added. Within the context of this embodiment, the addition of water may be carried out at the same elevated temperature as the dissolving step. Further, it is found that in some embodiments, adding the water slowly is particularly useful.

Next, the solution may be cooled. In some embodiments, it is found that cooling the solution to about room temperature (i.e., about 20° C. to about 30 ° C.) is particularly useful. In some embodiments, the cooling of the solution may result in a precipitate. In some embodiments, the solution is also stirred. Stirring may be carried out for any length of time to yield a precipitate. For example, in some embodiments, stirring is carried out for about 6 to about 10 hours.

Next, crystalline carfilzomib form M1 may be isolated. This may be achieved by methods well known in the art. For example, after precipitation of a solid, the resulting mixture may be filtered. The solid may be further processed, for example, by drying under vacuum to get crystalline carfilzomib form M1.

In some embodiments, the above procedure (i.e., steps a) through d)) may repeated to increase the purity of crystalline carfilzomib form M1.

In some embodiments, crystalline carfilzomib form M1, prepared by methods disclosed herein may exhibit long term stability. For example, some samples of crystalline carfilzomib form M1 have exhibited stability (measured by PXRD) when stored at 2° C. to 8° C. for one year.

Another aspect of the present invention provides a method for the preparation of amorphous carfilzomib.

In one embodiment, amorphous carfilzomib may be prepared by a process that includes the following steps:
a) dissolving crystalline carfilzomib in an alcohol solvent at about 20° C.-35° C. to get a solution;
b) combining the solution with water;
c) cooling the solution; and
d) isolating amorphous carfilzomib.

According to this embodiment, crystalline carfilzomib form M1 may be dissolved in an alcohol solvent at temperatures of about 20° C. to about 35° C. Within the context of this embodiment, the alcohol solvent may be, for example, methanol, ethanol, isopropanol, butanol, or mixtures thereof.

Next, the solution may be combined with water. This may be carried out by adding water to the carfilzomib solution or adding the carfilzomib solution to water.

Next, the solution may be cooled. In some embodiments, it is found that cooling the solution to about 0° C. to about 10° C. is useful. In some particularly useful embodiments, the solution is cooled to about 0° C. to about 5° C. In some embodiments, cooling the solution may result in precipitation of a solid, which may be carfilzomib.

Next, amorphous carfilzomib may be isolated. This may be carried out by methods well known in the art. For example, after precipitation of a solid, the resulting mixture may be filtered. The solid may be further processed, for example, by drying under vacuum to get amorphous carfilzomib. In some embodiments, it is found that treating the final solid with n-heptane is useful for removing remaining traces of the alcohol solvent.

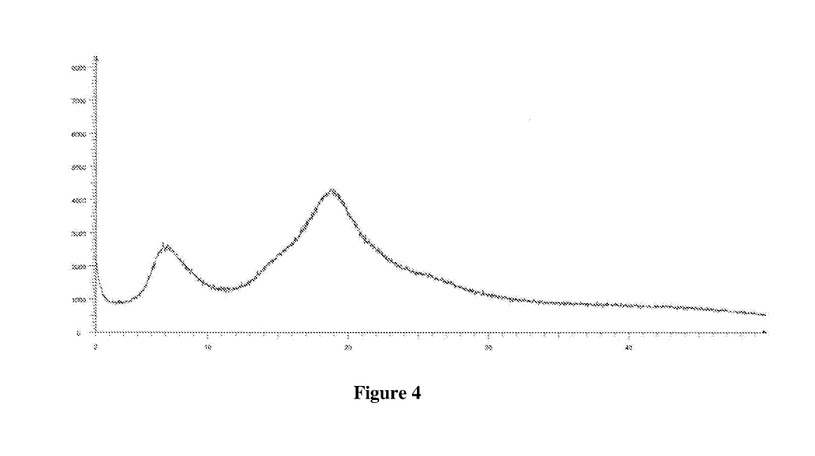

The amorphous carfilzomib prepared by methods disclosed herein may be characterized as amorphous by PXRD. For example, FIG. 4 shows one such PXRD pattern collected by PXRD analysis of amorphous carfilzomib.

Carfilzomib may be prepared as shown in scheme-1.

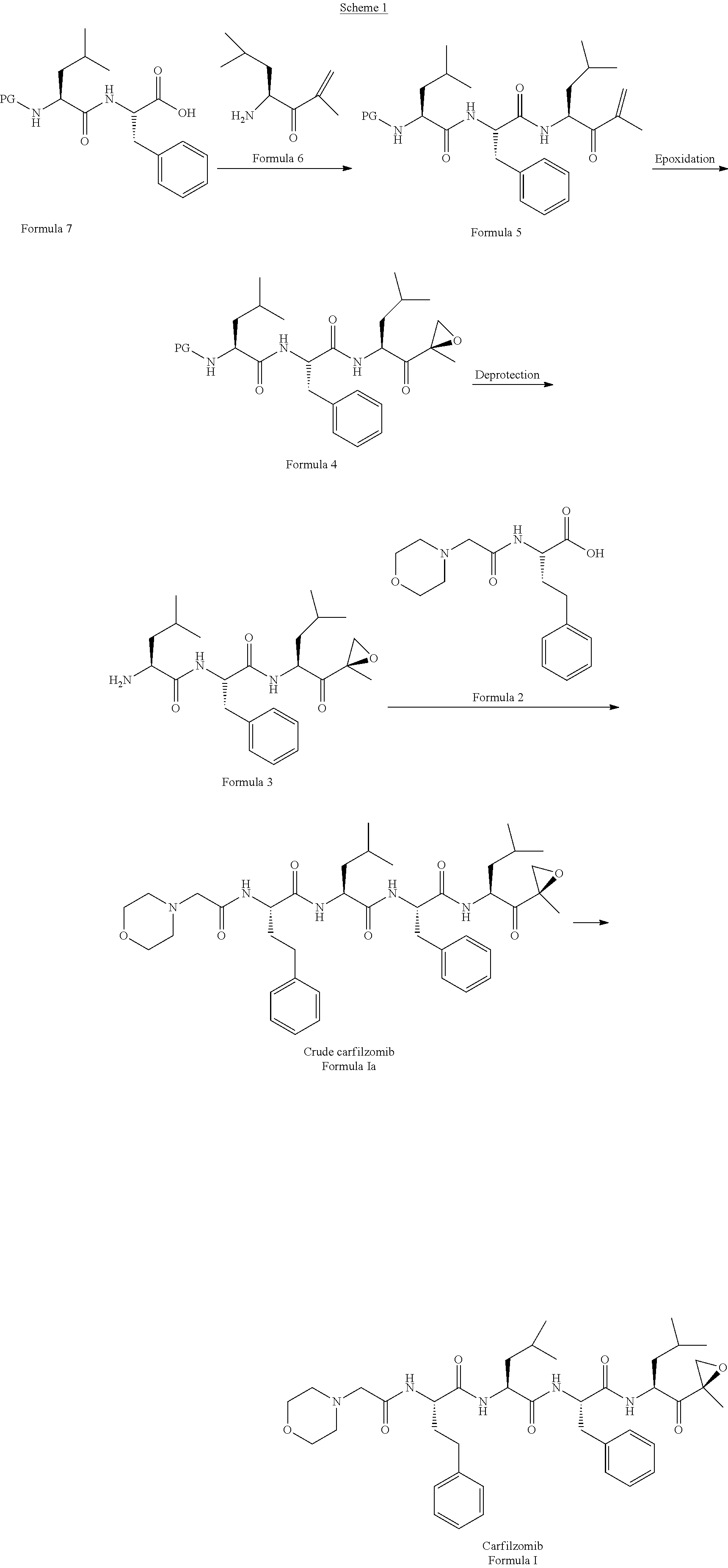

Within the context of Scheme 1, formulas 2 and 7may be prepared by processes disclosed in the prior art, for example, in IP.com disclosure number IPCOM000239813D.

The general mechanisms and chemistries represented in Scheme I may be carried out by methods well known in the art, for example, such as those disclosed in WO2009045497A1. One of skill in the art would be able to undertake reactions in Scheme 1 using well-known reaction conditions and reagents to be able to produce carfilzomib.

Within the context of scheme I, “PG” is an amine protecting group. Examples of suitable amine protecting groups, as well as suitable conditions for protecting and deprotecting, can be found in prior art, such as J. F. W. McOmie, “Protective Groups in Organic Chemistry”, Plenum Press, London and New York 1973; T. W. Greene and P. G. M. Wuts, “Protective Groups in Organic Synthesis”, Third edition, Wiley, New York 1999; “The Peptides”; Volume 3 (editors: E. Gross and J. Meienhofer), Academic Press, London and New York 1981; in “Methoden der organischen Chemie”, Houben-Weyl, 4th edition, Vol. 15/1, Georg Thieme Verlag, Stuttgart 1974; H.-D. Jakubke and H. Jescheit, “Aminosauren, Peptide, Proteine”, Verlag Chemie, Weinheim, Deerfield Beach, and Basel 1982; and Jochen Lehmann, “Chemie der Kohlenhydrate: Monosaccharide and Derivate”, Georg Thieme Verlag, Stuttgart 1974. In particularly useful embodiments, the protecting group is a tert-butyloxycarbonyl (BOC) group or a carboxybenzyl (Cbz) group.

The process for the preparation of crystalline carfilzomib form M1 disclosed herein may be advantageous over prior art processes. For example, purification of the final crystalline carfilzomib form M1 product may be achieved through crystallization from a solvent, without using any preparative HPLC. As such, the processes disclosed herein, using crystallization, may achieve a higher yield of crystalline carfilzomib form M1 by avoiding yield loss common in preparative chromatographic purification.

Crystalline carfilzomib form M1, prepared by methods disclosed herein may exhibit certain advantageous properties when compared to other forms of carfilzomib. For example, samples of crystalline carfilzomib form M1 prepared by methods disclosed herein were found to have a purity greater than 99.5%. In some samples, individual impurities were present at levels less than 0.1%. Further, samples of crystalline carfilzomib form M1 prepared by processes disclosed herein were found to be stable after one year (measured by PXRD) storage at 2° C. to 8° C.

In another aspect, the present disclosure provides a pharmaceutical composition containing carfilzomib and one or more pharmaceutically acceptable excipient. In some embodiments cyclodextrins, such as sulfobutylether beta-cyclodextrin, hydroxypropyl-beta-cyclodextrin, and the like is used as an excipient.

Within the context of this invention, the amorphous carfilzomib or crystalline carfilzomib form M1 may be formulated in to a dosage form suitable for intravenous delivery upon reconstitution with sterile water. Vials suitable for reconstitution may contain carfilzomib as a solid, including dosages of 30 mg to 60 mg per vial. In particularly useful embodiments, vials containing 30 mg or 60 mg are prepared. Such formulations may be useful in the treatment of multiple myeloma. Formulations of carfilzomib are particularly useful for patients having multiple myeloma who have received at least two prior therapies including bortezomib and an immunomodulatory agent and have demonstrated disease progression on or within sixty days of completion of the last therapy.

In view of the above description and the examples below, one of ordinary skill in the art will be able to practice the invention as claimed without undue experimentation. The foregoing will be better understood with reference to the following examples that detail certain procedures for the preparation of molecules, compositions, and formulations according to the present invention. All references made to these examples are for the purposes of illustration. The following examples should not be considered exhaustive, but merely illustrative of only a few of the many aspects and embodiments contemplated by the present disclosure.

### EXAMPLES

### Example 1: Preparation of Compound of Formula Ia

A compound of formula 2 (67.4 g, 1.2 eq.) was added to a solution of compound of formula 3(100 g, leq.) in ethyl acetate (2000 mL, 20 vol.). The mixture was cooled to 0-5° C. and N,N-diisopropylethylamine (163 mL, 5 eq.) was added slowly maintaining a pH of 8-9. Hydroxybenzotriazole (29.7 g, 1.2 eq.) and benzotriazol-1-yl-oxytripyrrolidinophosphonium hexafluorophosphate (114.4 g, 1.2 eq.) were added lot wise over 3-6 hours, stirring and maintaining the temperature at 0-5° C. and the pH at 8-9. Every hour, samples were collected and analyzed by HPLC to check for unreacted compound of formula 3. After completion of the reaction(when formula 3 levels were not more than 0.5%), 10% NaHCO₃ (1000 mL, 10 vol.) was added and the mixture was stirred at 25-30° C. for 30 minutes. The aqueous and organic layers were separated and the aqueous layer extracted with ethyl acetate (2×1000 mL, 2×10 vol.).The ethyl acetate layers were combined and washed sequentially with 10% NaHCO₃ (1000 mL, 10 vol.), water (1000 mL, 10 vol.), then brine (1000 mL, 10 vol.). The ethyl acetate layer was distilled to get a crude product.

The crude product was dissolved in methanol (800 mL, 8 vol.) and the obtained solution was added to water (20 L, 200 vol.) at 0-5° C. under vigorous stirring over 2-3 hours, maintaining the temperature at 0-5° C. The solution was filtered to obtain the precipitated solid, which was washed with water (2×1280 mL, 2×10 vol.).The solid was suck dried then dried under vacuum at 25-30° C. for 10-12 hours.

The solid was then taken in n-heptane (1000 mL, 10 vol.) at 25-30° C. and stirred for 2-4 hours. The solution was vacuum filtered and the obtained solid was washed with n-heptane (130 mL, 2 vol.). The solid was then vacuum dried in a vacuum oven at 40-45° C. for 10-12 hours until the moisture content was less than 5.0%. The solid resulting was a compound of formula Ia.

Yield: 113-125 g, 113-123%; Purity of compound of Formula Ia: 92-98%.

### Example 2: Preparation of Crystalline Carfilzomibform M1

Carfilzomib of formula Ia (100 g) was added to acetonitrile (1500 mL, 15 vol.) was added at 40-45 ° C. and stirred to dissolve solids. Water (300 mL, 3 vol.) was added slowly at 40±5° C. while stirring. After stirring for 30 minutes at 40±5° C., the solution was cooled to 20-30° C. and stirred further at 20-30° C. for 6-8 hours. The solution was vacuum filtered to obtain a crystalline material which was washed with a 1:1 mixture of water and acetonitrile (100 mL, 10 vol.). The solid was vacuum dried at 35-40° C. to get a white solid.

Yield: 72.5 g, w/w=72.5%; Purity: 97-99%.

The above solid (72.5 g) was taken in acetonitrile (1088 mL, 15 vol.) at 20-30° C. and the mixture was heated to 40-45° C. to dissolve the solid completely. Water (544 mL, 7.5 vol.) was added slowly at 40-45° C. while the solution was stirred. The solution was stirred for 30 minutes, maintaining the temperature at 40-45° C. After 30 minutes, the solution was cooled to 20-30° C. and stirred further at 20-30° C. for 6-8 hours. The solution was filtered to isolate the precipitated solid, which was then washed with water (72.5 mL, 10 vol.). The solid was vacuum dried then further dried at 35-40° C. under vacuum for 10-12 hours until the moisture content was less than 5%.

Yield: 62.5 g, w/w=86%; Purity: 99.63%.

### Example 3: Preparation of Amorphous Carfilzomib

Solid crystalline carfilzomib form M1, prepared by the process in Example 2 (62.5 g), was taken in methanol (625 mL, 10 vol.). The mixture was stirred to get a clear solution, which was filtered through Whatman filter paper (pore size 0.45 μm). Water (18.750 L, 300 vol.) was added slowly at 0-5° C. under vigorous stirring, and the stirring and temperature were maintained for 2-3 hours. The solution was filtered to isolate a precipitated solid, which was washed with water (2×625 mL, 2×10 vol.). The solid was vacuum dried for 6-8 hours.

The solid was then taken in n-heptane (625 mL, 10 vol.) at 25-30° C. The solution was stirred for 2-4 hours then filtered under suction. The obtained solid was washed with n-heptane (125 mL, 2 vol.), vacuum dried, then dried in a vacuum oven at 40-45° C. for 10-12 hours until the moisture content was less than 1.8% to yield amorphous carfilzomib.

Yield: 47-55 g, w/w 47-55%; Purity:99.70%.

### Example 4: Preparation of Amorphous Carfilzomib

Solid crystalline carfilzomib form M1, prepared by the process in Example 2 (62.5 g), was taken in methanol (625 mL, 10 vol.) and stirred to get a clear solution. The solution was filtered through Whatman filter paper (pore size 0.45 μm). Water (18.750 L, 300 vol.) was added to a separate reaction vessel and the solution was cooled to 0-5° C. The methanol/carfilzomib solution was added the reaction vessel, maintaining the temperature at 0-5° C. under vigorous stirring. Stirring was continued for 2-3 hours maintaining the same temperature. The solution was filtered to isolate the precipitated solid, which was washed with water (2×625 mL, 2×10 vol.). The solid was dried for 6-8hours under suction.

The solid was then taken in n-heptane (625 mL, 10 vol.) at 25-30° C. The mixture was stirred for 2-4 hours then filtered under suction. The obtained solid was washed with n-heptane (125 mL, 2 vol.), vacuum dried, then dried in a vacuum oven at 40-45° C. for 10-12 hours until the moisture content was less than 1.8% to obtain amorphous carfilzomib.

Yield: 47-55 g, w/w=47-55%; Purity:99.62%.

1. Crystalline carfilzomib form M1.

2. The crystalline carfilzomib form M1 of claim 1 having a powder X-ray diffraction pattern comprising peaks at 6.33, 12.76, and 19.85±0.2° 2θ.

3. The crystalline carfilzomib form M1 according to claim 2 having a powder X-ray diffraction pattern further comprising peaks at 9.28, 12.56, 18.67, 19.04, and 22.78±0.2° 2θ.

4. A process for preparation of crystalline carfilzomib form M1 according to claim 1, comprising the steps of:
a) dissolving carfilzomib in an organic solvent at an elevated temperature to form a solution;
b) adding water to the solution;
c) cooling the solution; and
d) isolating crystalline carfilzomib form M1.

5. The process according to claim 4, wherein the organic solvent is selected from the group consisting of acetonitrile, toluene, and mixtures thereof.

6. The process according to claim 4, wherein the elevated temperature is 35° C. to 50° C.

7. A process for preparation of amorphous carfilzomib, comprising the steps of:
a) dissolving crystalline carfilzomib in an alcohol solvent to form a solution;
b) combining the solution with water;
c) cooling the solution; and
d) isolating amorphous carfilzomib.

8. The process according to claim 7, wherein the alcohol solvent is selected from the group consisting of methanol, ethanol, isopropanol, butanol, and mixtures thereof.

9. A pharmaceutical composition comprising amorphous carfilzomib.

10. A pharmaceutical composition comprising the crystalline carfilzomib form M1 according to claim 1.

11. The pharmaceutical composition according to claim 9, further comprising a pharmaceutically acceptable excipient.

12. The pharmaceutical composition of claim 11, wherein the pharmaceutically acceptable excipient is selected from the group consisting of cyclodextrins, such as sulfobutylether beta-cyclodextrin, hydroxypropyl-beta-cyclodextrin, and the like.

13. The pharmaceutical composition according to claim 10, further comprising a pharmaceutically acceptable excipient.
